(19) United States
(12) Patent Application Publication
(10) Pub. No.: US 2025/0161194 A1
(43) Pub. Date: May 22, 2025

(54) MOUTHWASH FORMULATIONS

(71) Applicant: GURUNANDA, LLC, Buena Park, CA (US)

(72) Inventor: Puneet Nanda, Beverly Hills, CA (US)

(73) Assignee: GURUNANDA, LLC, Buena Park, CA (US)

(21) Appl. No.: 18/957,053

(22) Filed: Nov. 22, 2024

Related U.S. Application Data
(60) Provisional application No. 63/602,330, filed on Nov. 22, 2023.

(51) Int. Cl. A61K 8/92; A61K 8/06; A61K 8/67; A61Q 11/00

(52) U.S. Cl. CPC A61K 8/922; A61K 8/06; A61K 8/67; A61K 8/678; A61Q 11/00

(57) ABSTRACT

A concentrated mouthwash formulation is provided herein including a blend of essential oils comprising peppermint oil, tee tree oil, and clove oil, wherein the concentrated mouthwash formulation is configured to be dispersed within a volume of water to produce a mouthwash solution.

### CROSS REFERENCE TO RELATED APPLICATIONS

This application claims the benefit of and priority to U.S. Provisional Application Ser. No. 63/602,330, titled “MOUTHWASH FORMULATIONS” and filed Nov. 22, 2023, the contents of which are hereby incorporated by reference in their entirety.

### FIELD

Mouthwashes as disclosed herein relate to mouthwashes and mouthwash formulations used for human oral hygiene and, more specifically, to concentrated mouthwash and concentrated mouthwash formulations that are specially developed to be dispensed in a small amount and combined with water prior to being used by a person.

### BACKGROUND

The existence and use of mouthwash and mouthwash formulations that are provided in the form of a liquid compositions ready for use, mostly provided in the form of plastic bottle containers, are known. Such known mouthwashes contain certain ingredients that are provided in the form of an aqueous or alcohol-based solutions that are packaged in single-use large plastic bottles, of 16 ounces or more, to facilitate use by a person by simply dispensing a portion of the bottle contents in a small cup or the like and then placing or pouring in the person's mouth for use.

An issue that exists with such known mouthwashes is that once the contents of the single-use plastic container bottle are used the plastic bottle is thrown away. It is known that plastic is a large component of trash that presents an environmental challenge in that it is not biodegradable and takes up a relatively large amount of space. Another issue that exists with such known mouthwashes is the smell, which may be medicinal in nature or have another type of smell that is not pleasant to the user. A still other issue that exists with such known conventional mouthwashes is the chemicals that are used and the fact that such chemicals make it into the oceans through the sewer presenting a toxic issue for aquatic life. Moreover, mouthwash may be contaminated by being consumed directly from the mouthwash container.

It is, therefore, desired that a mouthwash and mouthwash formulation be developed that helps to mitigate or eliminate the above-noted issues.

### SUMMARY

In various embodiments, a concentrated mouthwash formulation is provided comprising a blend of essential oils comprising peppermint oil, tee tree oil, and clove oil, wherein the concentrated mouthwash formulation is configured to be dispersed within a volume of water to produce a mouthwash solution.

### BRIEF DESCRIPTION OF THE DRAWING FIGURES

FIG. 1 illustrates a method of making concentrated mouthwash formulations according to various embodiments.

FIG. 2 illustrates a method of making concentrated mouthwash formulations according to various embodiments.

### DESCRIPTION

The following is a list of definitions for terms used herein. Unless defined otherwise, all technical and scientific terms used herein generally have the same meaning as commonly understood by one of ordinary skill in the art. In the event that there is a plurality of definitions for a term herein, those in this section prevail unless stated otherwise. Generally, the nomenclature used herein and the laboratory procedures in cytopathicity analysis, microbial analysis, organic, physical and inorganic chemistry, and dental clinical research are those well-known and commonly employed in the art.

As used herein, “about” will be understood by persons of ordinary skill in the art and will vary to some extent on the context in which it can be used. Generally, “about” encompasses a range of values that are plus/minus 10% of a reference value, unless specifically defined. For instance, “about 25%” encompasses values from 22.5% to 27.5%.

As used herein, “ambient conditions” means approximately room temperature (e.g., 20-35° C.) and relative humidity of approximately <70%.

As used herein, “anti-microbial” means the property of inactivating or killing microorganisms, such as viruses, bacteria, algae, yeast, and fungi. As used herein, “biocidal” also refers to the effect of a composition as a treatment for reduction of bacterial or fungal or ither microbial growth or overgrowth in fluids or biofilm which may be associated with alleviating a diseased condition or state.

As used herein, “biostatic”, “bacteriostatic”, fungistatic” or synonymous terms means the property of arresting the growth of microorganisms, such as bacteria, algae, yeast and fungi. As used herein, “biostatic” means to the effect of a composition in maintaining the polymicrobial mixture of a fluid or a biofilm, as in maintaining the oral ecology so that one or more organisms have not overgrown to enable inflection and disease. Compositions with biostatic attributes are useful in health maintenance, wellness and prevention of infection and disease.

As used herein, “essentially free” means a composition which is comprised of very low levels, below detection levels of commonly used analytical methods, of a specific ingredient or compound or molecule.

As used herein, “substantially free of” means no more than 0.5% by weight of a given material.

Mouthwashes and mouthwash formulations as disclosed herein are unique in that they are formed of a combination of 100 percent pure and natural different essential oils that is provided in the form of a liquid concentrate. In an example, the concentrated mouthwash formulation may be provided in a small bottle container such as one comprising a dropper or the like to facilitate dispensing a small quantity of the liquid concentration into a volume of water for use as a mouthwash by a person. In an example embodiment, the container may be provided in the form of a small bottle having a dropper forming a lid of the bottle to facilitate easy use and dispensing by a user. In that regard, as used herein, a concentrated mouthwash formulation refers to the concentrated mouthwash formulation prior to being mixed with water for use by a person. In various embodiments, concentrated mouthwash formulations may comprise water, ethanol, or other hydrophilic compounds, but such concentrated mouthwash formulations are not intended to be used by a person without first being mixed with additional water. In various embodiments, concentrated mouthwash formulations do not contain water or ethanol.

Concentrated mouthwash formulations as disclosed herein may comprise and are not limited to a combination of the following ingredients that are provided in no specific order; peppermint, spearmint, cardamom, fennel, tea tree, oregano, clove, jasmine, and certain vitamins such as vitamin E, vitamin D, vitamin D3, and vitamin K2. Other ingredients may also be used with are described in detail below.

### Essential Oils

### Peppermint

In various embodiments, concentrated mouthwash formulations as disclosed herein may comprise peppermint. Peppermint is useful because it helps to freshen the breath, invigorate the mouth, and provide certain desired antibacterial benefits. In various embodiments, the peppermint is provided in the form of an essential oil. In an example, the peppermint essential oil may comprise approximately 100 percent by weight (i.e., pure) peppermint oil. In various embodiments, mouthwash formulations as disclosed herein may comprise from about 4 to 12 percent by weight peppermint oil based on the total weight of the concentrated mouthwash formulation. In a preferred embodiment, concentrated mouthwash formulations as disclosed herein may comprise from about 5 to 8 percent by weight peppermint oil, and in a most preferred embodiment approximately 6.13 percent by weight peppermint oil, based on the total weight of the concentrated mouthwash formulation.

### Spearmint

In an various embodiments, concentrated mouthwash formulations as disclosed herein may comprise spearmint. Spearmint is useful because it helps to freshen the breath, invigorate the mouth, and provide certain antibacterial, antimicrobial, and anti-inflammatory properties. In various embodiments, the spearmint is provided in the form of an essential oil. In an example, the spearmint essential oil may comprise approximately 100 weight percent (i.e., pure) spearmint oil. In various embodiments, mouthwash formulations as disclosed herein may comprise from about up to about 5 percent by weight spearmint oil based on the total weight of the concentrated mouthwash formulation. In a preferred embodiment, mouthwash formulations as disclosed herein may comprise from about 0.1 to 1 percent by weight spearmint oil, and in a most preferred embodiment approximately 0.66 percent by weight spearmint oil, based on the total weight of the concentrated mouthwash formulation.

### Cardamom

In various embodiments, concentrated mouthwash formulations as disclosed herein may comprise cardamom. Cardamom is useful because it helps to freshen the breath, invigorate the mouth, has certain desired antimicrobial properties, and helps fight tooth and gum decay. In various embodiments, the cardamom is provided in the form of an essential oil. In various embodiments, the cardamom essential oil may comprise approximately 100 percent by weight (i.e., pure) cardamom oil. In various embodiments, mouthwash formulations as disclosed herein may comprise up to about 3 percent by weight of cardamom oil based on the total weight of the concentrated mouthwash formulation. In a preferred embodiment, mouthwash formulations as disclosed herein may comprise from about 0.5 to 2 percent by weight cardamom oil, and in a most preferred embodiment approximately 1.2 percent by weight cardamom oil based on the total weight of the concentrated mouthwash formulation.

### Fennel

In various embodiments, concentrated mouthwash formulations as disclosed herein may comprise fennel. Fennel is useful because it helps to freshen the breath and invigorate the mouth. In various embodiments, the fennel is provided in the form of an essential oil. In an example, the fennel essential oil may comprise approximately 100 percent by weight (i.e., pure) fennel oil. In various embodiments, mouthwash formulations as disclosed herein may comprise up to about 3 percent by weight fennel oil based on the total weight of the concentrated mouthwash formulation. In a preferred embodiment, mouthwash formulations as disclosed herein may comprise from about 0.1 to 1 percent by weight fennel oil, and in a most preferred embodiment approximately 0.5 percent by weight fennel oil, based on the total weight of the concentrated mouthwash formulation.

### Tea Tree

In various embodiments, concentrated mouthwash formulations as disclosed herein may comprise tea tree. Tea tree is useful because it helps to support gum health, reduce inflammation, and provide certain desired antimicrobial properties. In an example, the tea tree is provided in the form of an essential oil. In various embodiments, the tea tree essential oil may comprise approximately 100 percent by weight (i.e., pure) tea tree oil. In an example, mouthwash formulations as disclosed herein may comprise less than about 4 percent by weight, and from about 0.05 to 3 percent by weight tea tree oil based on the total weight of the concentrated mouthwash formulation. In a preferred embodiment, mouthwash formulations as disclosed herein may comprise from about 0.1 to 1 percent by weight tea tree oil, and in a most preferred embodiment approximately 0.5 percent by weight tea tree oil, based on the total weight of the concentrated mouthwash formulation.

### Oregano

In various embodiments, concentrated mouthwash formulations as disclosed herein may comprise oregano. Oregano is useful because it helps to support gum health, reduce inflammation, and provide certain desired antibacterial and antimicrobial properties. In various embodiments, the oregano is provided in the form of an essential oil. In an example, the oregano essential oil may comprise approximately 100 percent by weight (i.e., pure) oregano oil. In various embodiments, mouthwash formulations as disclosed herein may comprise up to about 3 percent by weight of oregano oil based on the total weight of the concentrated mouthwash formulation. In a preferred embodiment, mouthwash formulations as disclosed herein may comprise from about 0.1 to 1 percent by weight oregano oil, and in a most preferred embodiment approximately 0.22 percent by weight oregano oil, based on the total weight of the concentrated mouthwash formulation.

### Clove

In various embodiments, concentrated mouthwash formulations as disclosed herein may comprise clove. Clove is useful because it helps to support gum health, reduce inflammation, and provide certain desired antibacterial, antifungal, antimicrobial, and antioxidant properties. In various embodiments, the clove is provided in the form of an essential oil. In an example, the clove essential oil may comprise approximately 100 percent by weight (i.e., pure) clove oil. In various embodiments, mouthwash formulations as disclosed herein may comprise less than about 4 percent by weight, and from about 0.2 to 3 percent by weight clove oil based on the total weight of the concentrated mouthwash formulation. In a preferred embodiment, mouthwash formulations as disclosed herein may comprise from about 0.5 to 1.5 percent by weight clove oil, and in a most preferred embodiment approximately 1 percent by weight clove oil, based on the total weight of the concentrated mouthwash formulation.

### Jasmine

In various embodiments, concentrated mouthwash formulations as disclosed herein may comprise Jasmine. Jasmine is useful because it produces a natural fragrance that helps to improve fragrance and the flavor profile. In various embodiments, the jasmine is provided in the form of an essential oil. In an example, the jasmine essential oil may comprise approximately 100 percent by weight (i.e., pure) jasmine oil. In various embodiments, mouthwash formulations as disclosed herein may comprise up to about 3 percent by weight of jasmine oil based on the total weight of the concentrated mouthwash formulation. In a preferred embodiment, mouthwash formulations as disclosed herein may comprise from about 0.01 to 1 percent by weight jasmine oil, and in a most preferred embodiment approximately 0.022 percent by weight jasmine oil, based on the total weight of the concentrated mouthwash formulation.

While certain types of essential oil ingredients that may be used to make mouthwash formulations as disclosed herein have been specifically described, it is to be understood that other essential oils in addition to those specifically described may be used if they provide certain beneficial properties to a user in the context of a mouthwash formulation end use application, and that such other essential oil ingredients are intended to be within the scope of mouthwash formulations as disclosed herein.

### Vitamins

### Vitamin E

In various embodiments, concentrated mouthwash formulations as disclosed herein may comprise one or more different types of vitamins. Vitamin E is useful to support oral and bone health, and to provide certain desired anti-inflammatory and antioxidant properties. In various embodiments, the vitamin E is provided in the form of tocopheryl acetate. In various embodiments, mouthwash formulations as disclosed herein may comprise from about 1 to 10 mg of vitamin E per usage amount of from about 0.2 to 0.3 mL of the mouthwash formulation. In a preferred embodiment, mouthwash formulations as disclosed herein may comprise from about 2 to 4 mg of vitamin E per usage of from about 0.2 to 0.3 mL of the mouthwash formulation.

### Vitamin D

In various embodiments, concentrated mouthwash formulations as disclosed herein may comprise one or more different types of vitamins. Vitamins D and D3 are useful to support oral and bone health. In various embodiments, the vitamin D is provided in the form of Cholecalciferol. In various embodiments, mouthwash formulations as disclosed herein may comprise from about 5 to 50 international units (IU) per usage amount of from about 0.2 to 0.3 mL of the mouthwash formulation. In a preferred embodiment, mouthwash formulations as disclosed herein may comprise from about 5 to 15 IU of vitamin D per usage amount of from about 0.2 to 0.3 mL of the mouthwash formulation.

### Vitamin K/K2

In various embodiments, concentrated mouthwash formulations as disclosed herein may comprise one or more different types of vitamins. Vitamins K and K2 are useful to support oral and bone health. In an example, the vitamin K2 (menaquinone) is provided. In various embodiments, mouthwash formulations as disclosed herein may comprise from about 5 to 50 international units (IU) per usage amount of from about 0.2 to 0.3 mL of the mouthwash formulation. In a preferred embodiment, mouthwash formulations as disclosed herein may comprise from about 5 to 15 IU of vitamin K2 per usage amount of from about 0.2 to 0.3 mL of the mouthwash formulation.

### Other Ingredients

### Water

In various embodiments, concentrated mouthwash formulations as disclosed herein may comprise water. Water is provided in the form of purified water (e.g., USP grade purified water). Water is used as solvent. In various embodiments, mouthwash formulations as disclosed herein may comprise up to about 40 percent by weight water based on the total weight of the mouthwash formulation. In a preferred embodiment, mouthwash formulations as disclosed herein may comprise from about 10 to 20 percent by weight water, and in a most preferred embodiment approximately 12.50 percent by weight water, based on the total weight of the concentrated mouthwash formulation.

### Sweetening Agent

In various embodiments, concentrated mouthwash formulations as disclosed herein may comprise a sweetening agent. In an example, stevia may be used as the sweetening agent. Stevia is used as a low calorie, sugarless, natural/naturally derived sweetener. In various embodiments, mouthwash formulations as disclosed herein may comprise from about 1 to 7 percent by weight stevia based on the total weight of the concentrated mouthwash formulation. In a preferred embodiment, concentrated mouthwash formulations as disclosed herein may comprise from about 2 to 5 percent by weight stevia, and in a most preferred embodiment approximately 3.03 percent by weight stevia, based on the total weight of the concentrated mouthwash formulation.

### Humectant and Solvent

In various embodiments, concentrated mouthwash formulations as disclosed herein may comprise a humectant and solvent. In an example, propanediol is used as the humectant and solvent. In various embodiments, concentrated mouthwash formulations as disclosed herein may comprise up to about 40 percent by weight propanediol based on the total weight of the concentrated mouthwash formulation. In a preferred embodiment, concentrated mouthwash formulations as disclosed herein may comprise from about 20 to 30 percent by weight propanediol, and in a most preferred embodiment approximately 27 percent by weight propanediol, based on the total weight of the concentrated mouthwash formulation.

### Co-Solvent

In various embodiments, concentrated mouthwash formulations as disclosed herein may comprise a co-solvent, such as an organic solvent. In various embodiments, ethyl alcohol is used as the co-solvent and also acts as a preservative. In various embodiments, concentrated mouthwash formulations as disclosed herein may comprise up to about 30 percent by weight ethyl alcohol based on the total weight of the concentrated mouthwash formulation. In a preferred embodiment, concentrated mouthwash formulations as disclosed herein may comprise from about 10 to 20 percent by weight ethyl alcohol, and in a most preferred embodiment approximately 20 percent by weight ethyl alcohol, based on the total weight of the concentrated mouthwash formulation.

### Emulsifier and Solubilizer

In various embodiments, concentrated mouthwash formulations as disclosed herein may comprise an emulsifier and solubilizer. In various embodiments, polysorbate 80 is used as the emulsifier and solubilizer. In various embodiments, concentrated mouthwash formulations as disclosed herein may comprise from about 10 to 40 percent by weight polysorbate 80 based on the total weight of the concentrated mouthwash formulation. In a preferred embodiment, mouthwash formulations as disclosed herein may comprise from about 20 to 30 percent by polysorbate 80, and in a most preferred embodiment approximately 26.50 percent by weight polysorbate 80, based on the total weight of the concentrated mouthwash formulation. In various embodiments, concentrated mouthwash formulations as disclosed herein may comprise menthol in amount of between 0.1% to 0.002% based on the total weight of the concentrated mouthwash formulation. In various embodiments, concentrated mouthwash formulations as disclosed herein may comprise menthol in amount of between 0.009% to 0.02% based on the total weight of the concentrated mouthwash formulation. In various embodiments, concentrated mouthwash formulations as disclosed herein may comprise menthol in amount of between 0.01% to 0.015% based on the total weight of the concentrated mouthwash formulation. In various embodiments, concentrated mouthwash formulations as disclosed herein may comprise menthol in amount of 0.012% based on the total weight of the concentrated mouthwash formulation.

While certain types of other ingredients that may be used with the essential oils to make mouthwash formulations as disclosed herein have been specifically described, it is to be understood that other types of other ingredients in addition to those specifically described above may be used if they provide certain beneficial properties to a user in the context of a mouthwash formulation end use application or help to facilitate making the concentrated mouthwash formulations or enabling the concentrated mouthwash formulation to function in the manner intended, and that such other ingredients not specifically mention are intended to be within the scope of mouthwash formulations as disclosed herein.

### Method of Making the Concentrated Mouthwash Formulation

Fast and even dispersion of the concentrated mouthwash in water during actual use is achieved through the use of appropriate emulsifying and solubilizing ingredients, such as polysorbate 80 and respective amounts of the same as disclosed above, and through the use of co-solvents such as ethyl alcohol and amounts of the same as disclosed above, as used to make concentrated mouthwash formulations as disclosed herein. These ingredients help to reduce interfacial tension between aqueous and non-aqueous phases and help the concentrate disperse easily in the final mouthwash solution. The ratio of solubilizer to cosolvents and aqueous phase on the concentrated mouthwash formulation is also important in achieving a clear solution when the concentrated mouthwash formulation is diluted in water before use. In an example, the ingredients used to make the concentrated mouthwash formulation may be combined in any order or fashion. However, in an example embodiment when stevia is used as the sweetening agent it is desired that the stevia be solubilized first in an aqueous medium before adding the remaining ingredients.

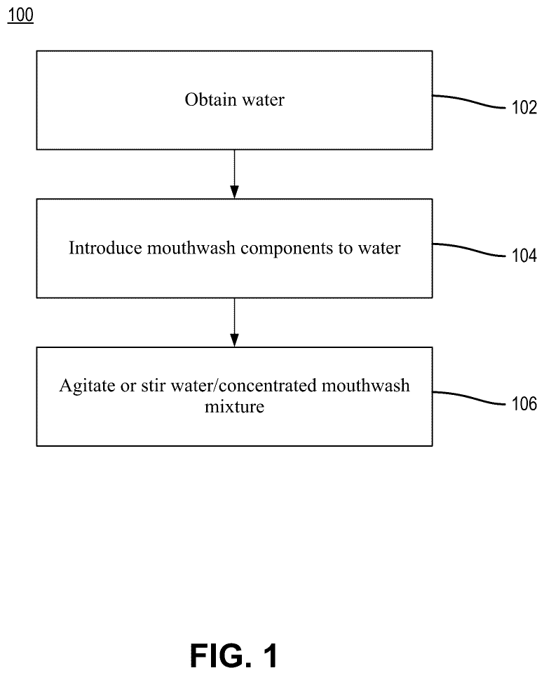

With reference to FIG. 1, method of making concentrated mouthwash formulation 100 is illustrated. At step 102, water is obtained. In that regard, water may be obtained in a vessel such as a bottle. At step 104, concentrated mouthwash components as disclosed herein is introduced to the water. Concentrated mouthwash components comprise one or more of clove oil, peppermint oil, clove oil, ethanol, propanediol, polysorbate 80, vitamin K, vitamin K2, vitamin D, vitamin D2, vitamin E, spearmint oil, cardamom oil, fennel oil, oregano oil, and jasmine oil. At step 106, the water/mouthwash mixture resulting from step 104 is agitated (e.g., shaken) or stirred to encourage mixture of the concentrated mouthwash and water.

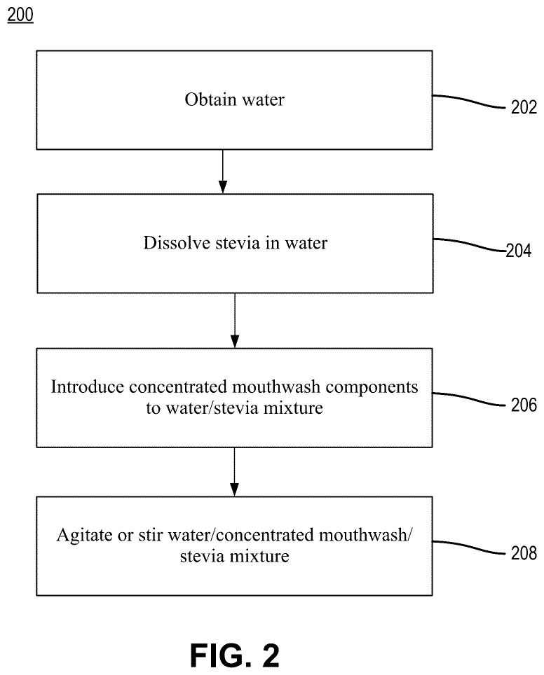

With reference to FIG. 2, method of making concentrated mouthwash formulation 200 is illustrated. At step 202, water is obtained. In that regard, water may be obtained in a vessel such as a bottle. At step 204, stevia is dissolved in the water. At step 206, concentrated mouthwash components as disclosed herein is introduced to the water/stevia mixture from step 204. Concentrated mouthwash components comprise one or more of clove oil, peppermint oil, clove oil, ethanol, propanediol, polysorbate 80, vitamin K, vitamin K2, vitamin D, vitamin D2, vitamin E, spearmint oil, cardamom oil, fennel oil, oregano oil, and jasmine oil. At step 208, the water/mouthwash/stevia mixture resulting from step 206 is agitated (e.g., shaken) or stirred to encourage mixture of the stevia, concentrated mouthwash and water.

### Method of Using the Concentrated Mouthwash Formulation

In various embodiments, concentrated mouthwash formulations as disclosed herein may be packaged in a small container bottle. In a particular example the container bottle may be plastic or glass, and in a particular example the container bottle is made from recycled plastic and is sized to contain approximately two ounces of the concentrated mouthwash formulation. In an example, the container may include a cap that is configured to include a dropper to enable a user to easily dispense drops of the concentrated mouthwash formulation into a volume of water, e.g., in a cup or the like that the user may use to place the mouthwash into their mouth. In an example, four to six drops of the concentrated mouthwash formulation into approximately 15 mL of water is sufficient to provide a final mouthwash solution for the user.

A feature of the concentrated mouthwash formulations as disclosed herein is that they are formed, in various embodiments, using a blend of 100 percent pure and natural essential oils. Further, the condensed nature of the mouthwash formulation enables a single two-ounce bottle to provide for about 300 mouthwash uses by a person, meaning that the single two ounce bottle may be mixed to form 300 doses of final mouthwash solution. When compared to conventional mouthwashes provided in single-use plastic containers of, for example, 16 ounces, the concentrated mouthwash formulation as disclosed herein when provided in a two ounce bottle effectively does the job of eight to ten of the conventional mouthwash 16 ounce plastic bottles, thereby drastically reducing the amount of plastic waste that is otherwise disposed and that adversely impacts the environment, as well as drastically reducing the shelf space associated with shipping and storing the same.

### Example 1: Formulations of Concentrated Mouthwash Formulations

Various compositions of concentrated mouthwash formulations are presented below. Concentrated mouthwash formulations and ingredients thereof are summarized in Table 1.

TABLE 1
Concentrated Mouthwash Formulations

| Ingredient | A | B | C | D | E | F | G | H |
|---|---|---|---|---|---|---|---|---|
| Aqueous | water | water | water | water | water | water | water | water |
| Solvent | | | | | | | | |
| Essential | peppermint | peppermint | peppermint | peppermint | peppermint | peppermint | peppermint | peppermint |
| Oil | oil, tea | oil, tea | oil, tea | oil, tea | oil, tea | oil, tea | oil, tea | oil, tea |
| | tree oil, | tree oil, | tree oil, | tree oil, | tree oil, | tree oil, | tree oil, | tree oil, |
| | clove oil | clove oil, | clove oil, | clove oil, | clove oil, | clove oil, | clove oil, | clove oil |
| | | spearmint | cardamom | fennel oil | oregano | jasmine | spearmint | |
| | | oil | oil | | oil | oil | oil, | |
| | | | | | | | cardamom | |
| | | | | | | | oil, | |
| | | | | | | | fennel oil, | |
| | | | | | | | oregano oil, | |
| | | | | | | | and jasmine | |
| | | | | | | | oil | |
| Humectant | propanediol | propanediol | propanediol | propanediol | propanediol | propanediol | propanediol | propanediol |
| & Solvent | | | | | | | | |
| Co- | ethanol | ethanol | ethanol | ethanol | ethanol | ethanol | ethanol | none |
| solvent | | | | | | | | |
| Vitamins | Vitamin E, | Vitamin E, | Vitamin E, | Vitamin E, | Vitamin E, | Vitamin E, | Vitamin E, | Vitamin E, |
| | vitamin D3, | vitamin D3, | vitamin D3, | vitamin D3, | vitamin D3, | vitamin D3, | vitamin D3, | vitamin D3, |
| | vitamin K2 | vitamin K2 | vitamin K2 | vitamin K2 | vitamin K2 | vitamin K2 | vitamin K2 | vitamin K2 |
| Sweetener | Stevia | Stevia | Stevia | Stevia | Stevia | Stevia | Stevia | None |
| Emulsifier | polysorbate | polysorbate | polysorbate | polysorbate | polysorbate | polysorbate | polysorbate | polysorbate |
| & | 80 | 80 | 80 | 80 | 80 | 80 | 80 | 80 |
| Solubilizer | | | | | | | | |

TABLE 2
Concentrated Mouthwash Formulations

| Ingredient | AA | BB | CC | DD | EE | FF | GG | HH |
|---|---|---|---|---|---|---|---|---|
| Aqueous | water | water | None | None | None | None | None | None |
| solvent | | | | | | | | |
| Essential | peppermint | peppermint | peppermint | peppermint | peppermint | peppermint | peppermint | peppermint |
| Oil | oil, tea | oil, tea | oil, tea | oil, tea | oil, tea | oil, tea | oil, tea | oil, tea |
| | tree oil, | tree oil, | tree oil, | tree oil, | tree oil, | tree oil, | tree oil, | tree oil, |
| | clove oil | clove oil | clove oil, | clove oil, | clove oil, | clove oil, | clove oil, | clove oil |
| | | | cardamom | fennel oil | oregano | jasmine | spearmint | |
| | | | oil | | oil | oil | oil, | |
| | | | | | | | cardamom | |
| | | | | | | | oil, fennel | |
| | | | | | | | oil, oregano | |
| | | | | | | | oil, and | |
| | | | | | | | jasmine oil | |
| Humectant | propanediol | propanediol | propanediol | propanediol | propanediol | propanediol | propanediol | propanediol |
| and | | | | | | | | |
| Solvent | | | | | | | | |
| Co- | ethanol | None | ethanol | ethanol | ethanol | ethanol | ethanol | ethanol |
| solvent | | | | | | | | |
| Vitamins | Vitamin E, | Vitamin E, | Vitamin E, | Vitamin E, | Vitamin E, | Vitamin E, | Vitamin E, | Vitamin E, |
| | vitamin D3, | vitamin D3, | vitamin D3, | vitamin D3, | vitamin D3, | vitamin D3, | vitamin D3, | vitamin D3, |
| | vitamin K2 | vitamin K2 | vitamin K2 | vitamin K2 | vitamin K2 | vitamin K2 | vitamin K2 | vitamin K2 |
| Sweetener | None | None | Stevia | Stevia | Stevia | Stevia | Stevia | Stevia |
| emulsifier | polysorbate | polysorbate | polysorbate | polysorbate | polysorbate | polysorbate | polysorbate | polysorbate |
| and | 80 | 80 | 80 | 80 | 80 | 80 | 80 | 80 |
| solubilizer | | | | | | | | |

An exemplary formulation of concentrated mouthwash formulation A is below in TABLE 3.

TABLE 3

| Ingredients | | |
|---|---|---|
| Water | 10-20% | w/w |
| Peppermint Oil | 4-15% | w/w |
| Tea Tree Oil | Less than 4% w/w | |
| Clove Oil | Less than 4% w/w | |
| Ethanol | 10-20% | w/w |
| Stevia | 1-7% | w/w |
| Polysorbate 80 | 20-30% | w/w |
| propanediol | 20-30% | w/w |

| | |
|---|---|
| Vitamin E | 1-10 mg per 0.2-0.3 mL |
| | concentrated |
| | mouthwash formulation |
| Vitamin D3 | 5-50 IU per 0.2-0.3 mL |
| | concentrated |
| | mouthwash formulation |
| Vitamin K2 | 5-50 IU per 0.2-0.3 mL |
| | concentrated |
| | mouthwash formulation |

An exemplary formulation of concentrated mouthwash formulation BB is below in TABLE 4.

TABLE 4

| Ingredients | | |
|---|---|---|
| Water | 10-60% | w/w |
| Peppermint Oil | 4-15% | w/w |
| Tea Tree Oil | Less than 4% w/w | |
| Clove Oil | Less than 4% w/w | |
| Polysorbate 80 | 20-30% | w/w |
| propanediol | 20-30% | w/w |

| | |
|---|---|
| Vitamin E | 1-10 mg per 0.2-0.3 mL |
| | concentrated mouthwash |
| | formulation |
| Vitamin D3 | 5-50 IU per 0.2-0.3 mL |
| | concentrated mouthwash |
| | formulation |
| Vitamin K2 | 5-50 IU per 0.2-0.3 mL |
| | concentrated mouthwash |
| | formulation |

An exemplary formulation of concentrated mouthwash formulation HH is below in TABLE 5.

TABLE 5

| Ingredients | | |
|---|---|---|
| Peppermint Oil | 4-15% | w/w |
| Tea Tree Oil | Less than 4% w/w | |
| Ethanol | 10-30% | w/w |
| Clove Oil | Less than 4% w/w | |
| Polysorbate 80 | 20-30% | w/w |
| Stevia | 1-7 20-30% | w/w |
| propanediol | 20-30% | w/w |

| | |
|---|---|
| Vitamin E | 1-10 mg per 0.2-0.3 mL |
| | concentrated |
| | mouthwash |
| | formulation |
| Vitamin D3 | 5-50 IU per 0.2-0.3 mL |
| | concentrated |
| | mouthwash |
| | formulation |
| Vitamin K2 | 5-50 IU per 0.2-0.3 mL |
| | concentrated |
| | mouthwash |
| | formulation |

Although example concentrated mouthwash formulations comprising certain described ingredients have been disclosed in detail above, those skilled in the art will readily appreciate that many modifications are possible in the example concentrated mouthwash formulations without materially departing from the intent and purpose of the example concentrated mouthwash formulations as disclosed herein. Accordingly, all such modifications of concentrated mouthwash formulations are intended to be included within the scope of this disclosure as defined in the following claims.

What is claimed is:

1. A concentrated mouthwash formulation comprising a blend of essential oils comprising peppermint oil, tee tree oil, and clove oil, wherein the concentrated mouthwash formulation is configured to be dispersed within a volume of water to produce a final mouthwash solution.

2. The concentrated mouthwash formulation of claim 1, further comprising at least one additional essential oil selected from the group consisting of spearmint oil, cardamom oil, fennel oil, oregano oil, and jasmine oil.

3. The concentrated mouthwash formulation of claim 1, further comprising at least two additional essential oils selected from the group consisting of spearmint oil, cardamom oil, fennel oil, oregano oil, and jasmine oil.

4. The concentrated mouthwash formulation of claim 1, further comprising at least three additional essential oil selected from the group consisting of spearmint oil, cardamom oil, fennel oil, oregano oil, and jasmine oil.

5. The concentrated mouthwash formulation of claim 1, further comprising at least four additional essential oil selected from the group consisting of spearmint oil, cardamom oil, fennel oil, oregano oil, and jasmine oil.

6. The concentrated mouthwash formulation of claim 1, further comprising spearmint oil, cardamom oil, fennel oil, oregano oil, and jasmine oil.

7. The concentrated mouthwash formulation of claim 1, wherein the blend of essential oils comprises at least 4 percent by weight of the total weight of the concentrated mouthwash formulation.

8. The concentrated mouthwash formulation of claim 1, wherein the essential oils comprise from about 5 to 25 percent by weight of the total weight of the concentrated mouthwash formulation.

9. The concentrated mouthwash formulation of claim 1, further comprising one or more vitamins selected from the group consisting of vitamin E, vitamin D3 and vitamin K2.

10. The concentrated mouthwash formulation of claim 1, further comprising vitamin E, vitamin D3 and vitamin K2.

11. The concentrated mouthwash formulation of claim 1, further comprising water.

12. The concentrated mouthwash formulation of claim 1, further comprising a sweetening agent.

13. The concentrated mouthwash formulation of claim 11, wherein the sweetening agent is stevia.

14. The concentrated mouthwash formulation of claim 1, further comprising an emulsifier and solubilizer.

15. The concentrated mouthwash formulation of claim 1, wherein the emulsifier and solubilizer is polysorbate 80.

16. A concentrated mouthwash formulation comprising:
water;
essential oils comprising peppermint oil, tee tree oil, and clove oil; vitamin E;
vitamin D;
a sweetening agent; and
an emulsifier and solubilizer.

17. The concentrated mouthwash formulation of claim 16, further comprising one or more of spearmint oil, cardamom oil, fennel oil, oregano oil, and jasmine oil.

18. The concentrated mouthwash formulation of claim 16, further comprising a humectant and an organic solvent.

19. The concentrated mouthwash formulation of claim 18, wherein the humectant is propanediol and the organic solvent is ethyl alcohol.

20. The concentrated mouthwash formulation of claim 15, wherein the sweetening agent is stevia, and emulsifier and solubilizer is polysorbate 80.
